(19) United States
(12) Patent Application Publication
(10) Pub. No.: US 2021/0371781 A1
(43) Pub. Date: Dec. 2, 2021

(54) Hybrid Solid State-Submerged Fermentation Using a Matrix

(71) Applicant: Locus IP Company, LLC, Solon, OH (US)

(72) Inventors: Sean FARMER, North Miami Beach, FL (US); Ken ALIBEK, Solon, OH (US)

(21) Appl. No.: 16/965,802

(22) PCT Filed: Sep. 26, 2019
(86) PCT No.: PCT/US2019/053218

§ 371 Date: Jul. 29, 2020

Related U.S. Application Data
(60) Provisional application No. 62/738,610, filed on Sep. 28, 2018.

(51) Int. Cl. C12M 1/107; C12M 1/02; C12M 1/00; C12M 1/34; C12M 1/36

(52) U.S. Cl. CPC C12M 21/04; C12M 27/00; C12M 47/10; C12M 41/16; C12M 41/48; C12M 29/18

(57) ABSTRACT

The subject invention provides methods and systems for producing microbe-based compositions that can be used in the oil and gas industry, environmental cleanup, agriculture, and many other applications. More specifically, the subject invention provides methods and systems for producing microorganisms and/or growth by-products thereof using a hybrid solid state-submerged fermentation method, wherein a matrix comprised of a solid material covered in an alginate coating is formed inside a liquid fermentation vessel. In some embodiments, the solid material is a plurality of natural loofa sponges.

### CROSS-REFERENCE TO RELATED APPLICATION

This application claims priority to U.S. Provisional Patent App. No. 62/738,610, filed Sep. 28, 2018, which is incorporated herein by reference in its entirety.

### BACKGROUND OF THE INVENTION

Cultivation of microorganisms such as bacteria, yeast and fungi is important for the production of a wide variety of useful bio-preparations. These microbes and their by-products are useful in many settings, such as oil production; agriculture; remediation of soils, water and other natural resources; mining; animal feed; waste treatment and disposal; food and beverage preparation and processing; and human health.

Interest in microbial surfactants, i.e., biosurfactants, in particular, has been steadily increasing in recent years due to their diversity, environmentally-friendly nature, selectivity, and performance under extreme conditions. Biosurfactants have excellent surface and interfacial tension reduction properties, as well as other beneficial biochemical properties, which can be useful in a variety of applications.

Additionally, biosurfactants contribute to the formation of micelles, providing a physical mechanism to mobilize, for example, oil in a moving aqueous phase. Furthermore, biosurfactants accumulate at interfaces, thus reducing interfacial tension and leading to the formation of aggregated micellar structures in solution. Advantageously, the ability of biosurfactants to form pores and destabilize biological membranes permits their use as, for example, antimicrobial and hemolytic agents. Thus, there exists an enormous potential for the use of microbes in a broad range of industries.

One limiting factor in commercialization of microbe-based products has been the cost per propagule density, where it is particularly expensive and unfeasible to apply microbial products to large scale operations. This is partly due to the difficulties in cultivating efficacious microbial products on a large scale.

Two principle forms of microbe cultivation exist for growing bacteria, yeasts and fungi: submerged (liquid fermentation) and surface cultivation (solid-state fermentation (SSF)). Both cultivation methods require a nutrient medium for the growth of the microorganisms, but they are classified based on the type of substrate used during fermentation (either a liquid or a solid substrate). The nutrient medium for both types of fermentation typically includes a carbon source, a nitrogen source, salts and other appropriate additional nutrients and microelements.

In particular, SSF utilizes solid substrates, such as bran, bagasse, and paper pulp, for culturing microorganisms. One advantage to this method is that nutrient-rich waste materials can be easily recycled as substrates. Additionally, the substrates are utilized very slowly and steadily, so the same substrate can be used for long fermentation periods. Hence, this technique supports controlled release of nutrients. SSF is best suited for fermentation techniques involving fungi and microorganisms that require less moisture content; however, it is less suitable for organisms that require high water activity.

Submerged fermentation, on the other hand, is typically better suited for those microbes that require high moisture. This method utilizes free flowing liquid substrates, such as molasses and nutrient broth, into which bioactive compounds are secreted by the growing microbes. While submerged cultivation can be achieved relatively quickly, the substrates are utilized quite rapidly, thus requiring constant replenishment and/or supplementation with nutrients. Additionally, it requires more energy, more stabilization, more sterilization, more control of contaminants, and often a more complex nutrient medium than is required for SSF. Furthermore, transporting microorganisms produced by submerged cultivation can be complicated and costly, in addition to the difficulty for laborers to implement the process in the field, e.g., in a remote location where the product will be used.

Microbes have the potential to play highly beneficial roles in, for example, the oil and agriculture industries; however, methods are needed for making microbe-based products more readily available, and preferably in a form that can be produced in, or transported to, remote areas without loss of efficacy.

### BRIEF SUMMARY OF THE INVENTION

The present invention provides materials, methods and systems for producing microbe-based compositions that can be used in the oil and gas industry, agriculture, health care and environmental cleanup, as well as for a variety of other applications. Specifically, the subject invention provides materials, methods and systems for efficient cultivation of microorganisms and production of microbial growth by-products using a hybrid of solid state and submerged fermentation methods.

Embodiments of the present invention provide methods and systems for cultivating a wide variety of yeasts, fungi and bacteria.

In certain embodiments, the systems can be used for the production of fungi- and/or yeast-based compositions, including, for example, compositions comprising a Trichoderma sp., Starmerella bombicola, Wickerhamomyces anomalus, Meyerozyma guilliermondii, Saccharomyces cerevisiae, Lentinula edodes, Pleurotus ostreatus and/or Pseudozyma aphidis.

In some embodiments, the systems can be used for the production of bacteria-based compositions, including, for example, compositions comprising Bacillus spp., Pseudomonas spp., Rhodococcus spp., and/or Acinetobacter spp.

In preferred embodiments, the system of the subject invention comprises one high volume vessel. Preferably, the vessel is a tank made of metal or a metal allow, for example, stainless steel, although other materials, such as plastic, are also envisioned. The tank can have an opening at the top that can be sealed during operation and/or cleaning.

In one embodiment, the tank is a modified stainless steel intermediate bulk container (“IBC”). Advantageously, the subject reactor systems can be scaled depending on the intended use. For example, the tank can range in volume from a few gallons to thousands of gallons. In some embodiments, the tank can hold about 1 to about 1,500 gallons. In some embodiments, a plurality of reactor systems can be set up inside an enclosure or housing facility to produce even greater total volumes of fermentation products.

The system can be equipped with one or more of: pH stabilization capabilities, temperature controls, an automated system for running a steam sterilization cycle; an impeller, or other form of mixing device; an external circulation system; and an aeration system or an air compressor.

In one embodiment, the external circulation system comprises two highly efficient external loops comprising inline heat exchangers. In one embodiment, the heat exchangers are shell-and-tube heat exchangers. Each loop is fitted with its own circulation pump.

The two pumps transport liquid from the bottom of the tank at, for example, 250 to 400 gallons per minute, through the heat exchangers, and back into the top of the tank. Advantageously, the high velocity at which the culture is pumped through the loops helps prevent cells from caking on the inner surfaces thereof.

The loops can be attached to a water source and, optionally, a chiller, whereby the water is pumped with a flow rate of about 10 to 15 gallons per minute around the culture passing inside the heat exchangers, thus increasing or decreasing temperature as desired. In one embodiment, the water controls the temperature of the culture without ever contacting the culture.

The reactor system can further comprise an aeration system capable of providing filtered air to the culture. The aeration system can, optionally, have an air filter for preventing contamination of the culture. The aeration system can function to keep the air level over the culture, the dissolved oxygen (DO), and the pressure inside the tank, at desired (e.g., constant) levels.

In certain embodiments, the unit can be equipped with a unique sparging system, through which the aeration system supplies air. Preferably, the sparging system comprises stainless steel injectors that produce microbubbles. In an exemplary embodiment, the spargers can comprise from 4 to 10 aerators, comprising stainless steel microporous pipes (e.g., having tens or hundreds of holes 1 micron or less in size), which are connected to an air supply. The unique microporous design allows for proper dispersal of oxygen throughout the culture, while preventing contaminating microbes from entering the culture through the air supply.

In some embodiments, the reactor system is controlled by a programmable logic controller (PLC). In certain embodiments, the PLC has a touch screen and/or an automated interface. The PLC can be used to start and stop the reactor system, and to monitor and adjust, for example, temperature, DO, and pH, throughout fermentation.

The reactor system can be equipped with probes for monitoring fermentation parameters, such as, e.g., pH, temperature and DO levels. The probes can be connected to a computer system, e.g., the PLC, which can automatically adjust fermentation parameters based on readings from the probes.

In certain embodiments, the DO is adjusted continuously as the microorganisms of the culture consume oxygen and reproduce. For example, the oxygen input can be increased steadily as the microorganisms grow, in order to keep the DO constant at about 30% (of saturation).

The reactor system can also be equipped with a system for running a steam sterilization cycle before and/or after running the reactor system. In certain embodiments, the steam sterilization system is automated.

The reactor system can comprise an off-gas system to release air. De-foaming measures can also be employed to suppress foam production, such as mechanical anti-foam apparatuses or chemical or biochemical additives.

In one embodiment, the subject invention provides methods of cultivating microorganisms using a system according to embodiments of the subject invention, wherein the methods comprise a hybrid between solid state and submerged forms of fermentation. In general, the methods comprise preparing a fermentation matrix inside the subject system, submerging the matrix in liquid culture medium, inoculating the system with a microorganism, cultivating the microorganism, and harvesting the microorganism.

In certain embodiments, the methods of cultivation comprise the step of preparing a fermentation matrix inside the fermentation system vessel. In one embodiment, this comprises adding a solid material with ample surface area into the system until the material reaches a height that is about 50% to 90% of the tank height.

In one embodiment, the matrix is formed by stacking multiple layers comprising rows of natural loofa sponges into the tank of the subject system to form a three-dimensional solid matrix on which microorganisms can grow. A first layer of loofas can be placed on the interior base (i.e., floor) of the tank, preferably covering the entire area of the base. Then, additional layers can be stacked on top until the matrix reaches a desired height.

Preferably, the loofas are obtained from natural sources, such as the dried, cylindrical-shaped fruits of Luffa aegyptiaca and/or Luffa acutangula. Advantageously, the loofas have hundreds of fibrous layers, folds and crevices that allow for unhindered circulation of, e.g., nutrients and oxygen through the matrix, and provide a broader surface area on and in which the microorganisms can deposit and grow.

Similar solid materials, such as natural or synthetic sponges, synthetic loofas, and even bio-balls that are utilized in cleaning of aquariums, can also be utilized. In one embodiment, the solid material comprises whole and/or pieces of sea shells, or the shells of any hard-shell animal such as a mollusk or crustacean. For example, the solid material can be the empty shells of mussels, scallops, conches, oysters, clams and/or snails.

Next, the method comprises submerging the fermentation matrix in liquid culture medium, wherein the culture medium is added to the system using, for example, a peristaltic pump, and optionally, circulated through the system using the circulating pumps. In some embodiments, the amount of liquid culture medium added is enough to cover the matrix entirely. Preferably, however, the culture medium does not fill the reactor vessel entirely, as additional liquid must be added to the system during the inoculation step.

In certain embodiments, the culture medium comprises a protein source (e.g., yeast extract or corn peptone), a carbon source (e.g., glucose or molasses), salts, and other necessary vitamins, minerals and nutrients that are optimal for production of a certain microorganism and/or microbial growth by-product.

In one embodiment, the culture medium can comprise agar and/or alginate, which can provide a semi-solid adherent for enhanced microbial deposition and adhesion onto the matrix. In certain embodiments, an antimicrobial agent is added to the medium to prevent growth of a contaminating microorganism, such as an antibiotic.

In one embodiment, the method further comprises inoculating the system with a microorganism. Preferably, inoculation according to the subject methods comprises mixing a microbial inoculant (e.g., cells and/or spores) in filtered water and pumping the inoculant and water into the system. The inoculant, water, and culture medium can then be circulated throughout the system to ensure exposure to the various surfaces of the matrix and adhesion thereto. Circulation can be performed using, for example, the circulation pumps and external loops, or simply by using a mixing device.

In one embodiment, the method further comprises cultivating the microorganism for a number of days until a desired cell concentration is achieved. In one embodiment, the microorganisms grow for 1 to 21 days, preferably from 1 to 14 days, even more preferably from 2 to 10 days.

In one embodiment, the method further comprises harvesting the microorganisms from the system. In certain embodiments, harvesting comprises pumping a mixture comprising water and a biosurfactant into the system and optionally, circulating the mixture throughout the system. Preferably, the water is filtered water and the biosurfactant is a sophorolipid (SLP).

In certain embodiments, the circulation of the mixture over and through the matrix provides enough agitation to detach the microorganisms from the matrix and into the liquid. In certain embodiments, the SLP helps to break the surface tension between the microorganisms and the matrix, thus providing further detachment of microorganisms from the matrix. Even further, air can be pumped into the liquid to provide further turbulence for detaching the culture.

After the microorganisms have been detached, the liquid in the system, comprising detached microorganisms, growth by-products, water and residual nutrients, can be drained from the system. If needed, the harvesting process can be repeated until all or most of the culture has been detached from the matrix and washed from the system.

In one embodiment, the subject invention also provides methods of producing a microbial growth by-product, wherein the method comprises cultivating a microorganism according to the subject methods and under conditions favorable for growth and metabolite production, and optionally, purifying the growth by-product. In specific embodiments, the growth by-product is a biosurfactant, an enzyme, biopolymer, acid, solvent, amino acid, nucleic acid, peptide, protein, lipid and/or carbohydrate.

In one embodiment, the subject invention provides a composition comprising at least one type of microorganism and/or at least one microbial metabolite produced by the microorganism. Preferably, the composition is produced according to the subject methods.

The microorganisms in the composition may be in an active or inactive form. The composition can be subjected to filtration, centrifugation, lysing, drying or processing by any known means depending upon the desired use. Alternatively, the composition can be utilized as is, in liquid form, without further processing.

Advantageously, the method and equipment of the subject invention reduce the capital and labor costs of producing microorganisms and their metabolites on a large scale. Furthermore, the subject invention provides a cultivation method that not only substantially increases the yield of microbial products per unit of nutrient medium but simplifies production in an environmentally-friendly manner using renewable substrates.

### DETAILED DESCRIPTION OF THE INVENTION

The present invention provides materials, methods and systems for producing microbe-based compositions that can be used in the oil and gas industry, agriculture, health care and environmental cleanup, as well as for a variety of other applications. Specifically, the subject invention provides materials, methods and systems for efficient cultivation of microorganisms and production of microbial growth by-products using a hybrid of solid state and submerged fermentation.

Embodiments of the present invention provide methods and systems for cultivating a wide variety of yeast, fungi and bacteria.

The system can comprise a vessel, such as a tank, pH stabilization capabilities and temperature controls. The system can also be equipped with an impeller, or other form of mixing device. The system can further comprise an aeration system or an air compressor and a sparging system through which the aeration system supplies air.

In one embodiment, the subject invention provides methods of cultivating microorganisms using the subject system, wherein the methods comprise a hybrid between solid state and submerged forms of fermentation. In general, the methods comprise preparing a fermentation matrix comprised of a solid material inside the subject system, submerging the matrix in liquid culture medium, inoculating the system with a microorganism, cultivating the microorganism, and harvesting the microorganism.

### Selected Definitions

As used herein, a “biofilm” is a complex aggregate of microorganisms, such as bacteria, wherein the cells adhere to each other and/or to a surface via an extracellular polysaccharide matrix. The cells in biofilms are physiologically distinct from planktonic cells of the same organism, which are single cells that can float or swim in liquid medium.

As used herein, the term “control” used in reference to a pest or other undesirable organism extends to the act of killing, disabling or immobilizing the pest or other organism, or otherwise rendering the pest or other organism substantially incapable of causing harm.

As used herein, “harvested” in the context of microbial fermentation refers to removing some or all of a microbe-based composition from a growth vessel.

As used herein, “intermediate bulk container,” “IBC” or “pallet tank” refers to a reusable industrial container designed for transporting and storing bulk substances, including, e.g., chemicals (including hazardous materials), food ingredients (e.g., syrups, liquids, granulated and powdered ingredients), solvents, detergents, adhesives, water and pharmaceuticals. Typically, IBCs are stackable and mounted on a pallet designed to be moved using a forklift or a pallet jack. Thus, IBCs are designed to enable portability.

As used herein, an “isolated” or “purified” nucleic acid molecule, polynucleotide, polypeptide, protein, organic compound such as a small molecule (e.g., those described below), or other compound is substantially free of other compounds, such as cellular material, with which it is associated in nature. For example, a purified or isolated polynucleotide (ribonucleic acid (RNA) or deoxyribonucleic acid (DNA)) is free of the genes or sequences that flank it in its naturally-occurring state. A purified or isolated polypeptide is free of the amino acids or sequences that flank it in its naturally-occurring state. A purified or isolated microbial strain is removed from the environment in which it exists in nature. Thus, the isolated strain may exist as, for example, a biologically pure culture, or as spores (or other forms of the strain) in association with a carrier.

In certain embodiments, purified compounds are at least 60% by weight the compound of interest. Preferably, the preparation is at least 75%, more preferably at least 90%, and most preferably at least 99%, by weight the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis.

A “metabolite” refers to any substance produced by metabolism (e.g., a growth by-product) or a substance necessary for taking part in a particular metabolic process. A metabolite can be an organic compound that is a starting material, an intermediate in, or an end product of metabolism. Examples of metabolites can include, but are not limited to, enzymes, toxins, acids, solvents, alcohols, proteins, carbohydrates, vitamins, minerals, microelements, amino acids, polymers, and surfactants.

As used herein, reference to a “microbe-based composition” means a composition that comprises components that were produced as the result of the growth of microorganisms or other cell cultures. Thus, the microbe-based composition may comprise the microbes themselves and/or by-products of microbial growth. The microbes may be in a vegetative state, in spore form, in mycelial form, in any other form of microbial propagule, or a mixture of these. The microbes may be planktonic or in a biofilm form, or a mixture of both. The by-products of growth may be, for example, metabolites (e.g., biosurfactants), cell membrane components, expressed proteins, and/or other cellular components. The microbes may be intact or lysed. The cells may be totally absent, or present at, for example, a concentration of at least 1×10⁴, 1×10⁵, 1×10⁶, 1×10⁷, 1×10⁸, 1×10⁹, 1×10¹⁰, 1×10¹¹, 1×10¹², 1×10¹³ or more CFU/ml of the composition.

The subject invention further provides “microbe-based products,” which are products that are to be applied in practice to achieve a desired result. The microbe-based product can be simply the microbe-based composition harvested from the microbe cultivation process. Alternatively, the microbe-based product may comprise further ingredients that have been added. These additional ingredients can include, for example, stabilizers, buffers, carriers (e.g., water or salt solutions), added nutrients to support further microbial growth, non-nutrient growth enhancers and/or agents that facilitate tracking of the microbes and/or the composition in the environment to which it is applied. The microbe-based product may also comprise mixtures of microbe-based compositions. The microbe-based product may also comprise one or more components of a microbe-based composition that have been processed in some way such as, but not limited to, filtering, centrifugation, lysing, drying, purification and the like.

As used herein, “surfactant” refers to a compound that lowers the surface tension (or interfacial tension) between two liquids or between a liquid and a solid. Surfactants act as detergents, wetting agents, emulsifiers, foaming agents, and dispersants. A “biosurfactant” is a surfactant produced by a living organism.

The transitional term “comprising,” which is synonymous with “including,” or “containing,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention. Use of the term “comprising” contemplates embodiments “consisting” and “consisting essentially” of the recited component(s).

Unless specifically stated or obvious from context, as used herein, the term “or” is understood to be inclusive. Unless specifically stated or obvious from context, as used herein, the terms “a,” “and” and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

All references cited herein are hereby incorporated by reference in their entirety.

### Hybrid Solid State and Submerged Fermentation System

In a specific embodiment, the system (also referred to as “unit” or “reactor”) of the subject invention comprises a reaction vessel. The vessel used according to the subject invention can be any fermenter or cultivation reactor for industrial use. The vessel can be a tank or a barrel or any other container. These vessel may be made of, for example, glass, polymers, metals, metal alloys, and combinations thereof.

The tank can have a sealable opening located, for example, at the top. The tank can also have one or more sight glasses for visual monitoring of the culture inside the tank.

Advantageously, the reactor system can be scaled depending on the intended use. For example, the tank can range in volume from a few gallons to thousands of gallons. In one embodiment, the ratio of tank width to height is 1:2 to 1:5.

In some embodiments, the tank can hold about 1 to about 1,500 gallons. In some embodiments, the tank can hold about 5 liters to 5,000 liters or more. Typically, the tank will be from 10 to 4,000 liters, and preferably from 100 to 2,500 liters.

In one exemplary embodiment, the tank has a volume of 550 gallons (about 2,082 liters) and measures about 4 to 5 feet in length and/or width, and about 5 to 6 feet in height.

In certain embodiments, when scaling up is desired, a plurality of systems can be connected to one another and utilized as one cascading system. For example, a battery of tanks can be set up in close proximity to one another and connected via tubing or piping. Excess liquid from one system can be released through the tubing or piping and supplanted into the next system and so on until all of the systems are filled. The fermentation cycles in each tank can be run simultaneously or can be staggered so as to provide continuous production and harvesting of microorganisms.

The system can be equipped with one or more of: pH stabilization capabilities, temperature controls, an automated system for running a steam sterilization cycle; an impeller, or other form of internal mixing device; an external circulation system; and an aeration system or an air compressor.

In one embodiment, the internal mixing device comprises a mixing motor located at the top of the tank. In one embodiment, the mixing motor rotates on a diagonal axis (e.g., an axis at 15 to 60° from vertical). The motor is rotatably attached to a metal shaft that extends into the tank and is fixed with an impeller to help propel liquid from the top of the tank to the bottom of the tank and to ensure efficient mixing and gas dispersion throughout the culture, as well as efficient mass exchange. In some embodiments, the shaft is fixed with two or more impellers.

In one embodiment, the impeller is a standard four-blade Rushton impeller. In one embodiment, the impeller comprises an axial flow aeration turbine and/or a small marine propeller. In one embodiment, the impeller design comprises customized blade shapes to produce increased turbulence.

In one embodiment, the system comprises an external circulation system, which doubles as a temperature control system. Advantageously, the external circulation system obviates the need for a double-walled tank, or an external temperature control jacket.

In one embodiment, the external circulation system comprises a first and a second highly efficient external loop comprising a first and a second inline 300K to 360K heat exchanger. Either or both of the loops can be located on either side of the tank and/or on the back of the tank.

In one embodiment, the heat exchangers are shell-and-tube heat exchangers. Each loop is fitted with its own 1-2 horsepower circulation pump.

The two pumps transport liquid from the bottom of the tank at, for example, 250 to 400 gallons per minute, through the first and second heat exchangers, and back into the tank at the top. Advantageously, the velocity at which the culture is pumped through the two loops helps prevent cells from caking on the inner surfaces thereof.

The first and second loops can be attached to a water source, and optionally, a chiller, whereby the water is pumped with a flow rate of about 10 to 15 gallons per minute around the culture passing inside the heat exchangers, thus increasing or decreasing temperature as desired. In some embodiments, the water is filtered through a water filter.

The heat exchangers can utilize an electric heater; however, for larger applications where heat is required, steam or hydrocarbon fuel can be utilized to generate heat. For example, steam input and/or a steam source can be connected to the heat exchangers.

The heat exchangers provide a closed system where the cooling water or steam used for temperature control do not contact the culture. Advantageously, the external circulation system can also be used to clean the reactor system in between cycles, wherein steam and/or hot water is circulated through the tank and the external loops for a time sufficient to remove cell matter and any other contaminants.

In one embodiment, the reactor system may be adapted to ensure maintenance of an appropriate fermentation temperature, particularly if the reactor system is being operated outdoors. In preferred embodiments, however, such adaptations are not necessary due to the use of the external circulation system. For example, the outside of the reactor system can be reflective to avoid raising the system temperature during the day if being operated outdoors. The reactor system can also be insulated so the fermentation process can remain at appropriate temperatures in low temperature environments. Any of the insulating materials known in the art can be applied including fiberglass, silica aerogel, ceramic fiber insulation, etc. The insulation (not shown) can surround any and/or all of the components of the system.

The reactor system can further comprise an aeration system. The aeration system can, optionally, have an air filter for preventing contamination of the culture. The aeration system can function to keep the air level over the culture, the DO, and the pressure inside the tank, at desired (e.g., constant) levels.

In certain embodiments, the reactor system can be equipped with a unique sparging system, through which the aeration system supplies air. In some embodiments, the sparging system is fixed at the bottom and/or along the inner sides of the tank.

Preferably, the sparging system comprises multiple aerators that produce microbubbles of air. In an exemplary embodiment, the sparging system comprises from 4 to 10 aerators, comprising stainless steel microporous pipes connected perpendicularly to a central air supply pipe. The microporous pipes comprise a plurality (e.g., tens to hundreds) of holes, through which air is injected into the culture in the form of microbubbles.

In preferred embodiments, the holes in the microporous pipes of the aerators are 1 micron in diameter or less, preferably about 0.01 to 0.5 micron, more preferably, about 0.1 to 0.2 micron. The unique microporous design allows for dispersal of oxygen throughout the culture. Furthermore, injection of air through submicron-sized holes prevents contaminating microbes from entering the culture through the aeration system and air supply.

In one embodiment, the impeller helps keep the microbubbles from coalescing into larger-sized bubbles.

The reactor system can be equipped with a system for running a steam sterilization cycle before and/or after running the reactor system. In certain embodiments, the steam sterilization system is automated.

The reactor system can comprise an off-gas system to release air. De-foaming measures can also be employed to suppress foam production, such as mechanical anti-foam apparatuses or addition of chemical or biochemical anti-foam additives.

In some embodiments, the reactor system is controlled by a programmable logic controller (PLC). In certain embodiments, the PLC has a touch screen and/or an automated interface. The PLC can be used to start and stop the reactor system, and to monitor and implement adjustments to, for example, temperature, DO, and pH, throughout fermentation. Desired measurements can be programmed into the computer prior to the reactor system being delivered to a site, or on-site prior to operation.

In one embodiment, the reactor system has functional controls/sensors/probes or may be capable of being connected to functional controls/sensors/probes for measuring cultivation parameters either automatically or manually. These parameters can include, for example, pH, DO, pressure, temperature, agitator shaft power, humidity, viscosity, microbial density and/or metabolite concentration.

The probes can be connected to a computer system, e.g., the PLC, which utilizes an electronic panel to implement adjustments to fermentation parameters based on readings from the probes. Adjustments can be made automatically or can be directed manually by a user.

The pH can be set to a specific number by a user or the computer can be pre-programmed to direct changes in the pH according to probe readings throughout the fermentation process. If the pH adjustment is to be done manually, pH measurement tools known in the art can be included with the system for manual testing.

The temperature can be set to a specific measurement by a user or the computer can be pre-programmed to direct changes in the temperature according to probe readings throughout the fermentation process. In certain embodiments, the temperature probe is a thermometer. The temperature measurements can be used to automatically or manually control the temperature control systems that are discussed above.

In certain embodiments, the DO is monitored and adjusted continuously as the microorganisms of the culture consume oxygen and reproduce. For example, in response to DO readings from the probes, the computer can direct the aeration system to keep the DO constant at about 30% (of saturation). In one embodiment, this can be achieved by cascade, where the amount of oxygen input is increased steadily as the microorganisms grow and consume greater amounts thereof.

In addition to monitoring and controlling temperature and pH, each reactor system may also have the capability for monitoring and controlling, for example, agitation, foaming, purity of microbial cultures, production of desired metabolites and the like. The reactor systems can further be adapted for remote monitoring of these parameters, for example with a tablet, smart phone, or other mobile computing device capable of sending and receiving data wirelessly.

In a further embodiment, the system may also be able to monitor the growth of microorganisms inside the vessel (e.g., measurement of cell number and growth phases). Alternatively, a daily sample may be taken from the vessel and subjected to enumeration by techniques known in the art, such as dilution plating technique.

In one embodiment, the reactor system is a mobile or portable bioreactor that may be provided for on-site production of a microbiological product including a suitable amount of a desired strain of microorganism. Because the microbiological product is generated on-site of the application, without resort to the bacterial stabilization, preservation, storage and transportation processes of conventional production, a much higher density of live microorganisms may be generated, thereby requiring a much smaller volume of the microorganism composition for use in the on-site application. This facilitates the mobility and portability of the system.

The reactor system can include a frame or a stand for supporting the apparatus components. The system can include wheels for moving the apparatus, as well as handles for steering, pushing and pulling when maneuvering the apparatus. Furthermore, the system can comprise forklift pockets for efficient transport using a forklift.

The reactor system can be suitable for transport on a pickup truck, a flatbed trailer, or a semi-trailer, or can even be configured onto the back of a flatbed truck, truck trailer and/or semi-trailer.

### METHODS

In certain embodiments, methods are provided for cultivating a wide variety of yeasts, fungi and bacteria using a system according to embodiments of the subject invention, wherein the methods comprise a hybrid between solid state and submerged forms of fermentation. The system can include all of the materials necessary for the fermentation (or cultivation) process, including, for example, equipment, sterilization supplies, and culture medium components, although it is expected that freshwater could be supplied from a local source and sterilized according to the subject methods.

In general, the methods comprise preparing a fermentation matrix inside the subject system, submerging the matrix in liquid culture medium, inoculating the system with a microorganism, cultivating the microorganism, and harvesting the microorganism.

In a specific embodiment, the method of cultivation first comprises sterilizing the subject fermentation reactors prior to preparation of the fermentation matrix. The tanks of the system may be disinfected or sterilized. The cultivation equipment such as the reactor/vessel may be separated from, but connected to, a sterilizing unit, e.g., an autoclave. The cultivation equipment may also have a sterilizing unit that sterilizes in situ before starting the inoculation, e.g., by using a steamer.

In certain embodiments, before fermentation, the tank can be washed with a hydrogen peroxide solution (e.g., from 1.0% to 4.0% hydrogen peroxide; this can be done before or after a hot water rinse at, e.g., 80-110° C.) to prevent contamination. In addition, or in the alternative, the tank can be washed with a commercial disinfectant, a bleach solution and/or a hot water or steam rinse.

In certain specific embodiments, the internal surfaces of the reactor (including, e.g., tanks, ports, spargers and mixing systems) can first be washed with a commercial disinfectant; then fogged (or sprayed with a highly dispersed spray system) with 1% to 4% hydrogen peroxide, preferably 3% hydrogen peroxide; and finally steamed at a temperature of about 105° C. to about 110° C., or greater.

In certain embodiments, the methods of cultivation comprise the step of preparing a fermentation matrix comprised of a solid material inside the system of the subject invention. In one embodiment, this comprises placing the solid material into the tank of the subject system to form a three-dimensional solid matrix on which microorganisms can grow.

In certain embodiments, the material comprises a plurality of loofa sponges. In a specific embodiment, the matrix comprises a plurality of layers made up of rows of loofa sponges aligned in parallel to one another. Preferably, the loofas (or “luffa”) are obtained from natural sources, such as the dried, oblong fruits of Luffa aegyptiaca and/or Luffa acutangula. Advantageously, when the fruit is dried and processed such that only the xylem fibers remain, the loofas comprise hundreds of fibrous layers, folds and crevices that allow for unhindered circulation of, e.g., nutrients and oxygen through the matrix, and provide a broader surface area on and in which the microorganisms can deposit and grow. When used as a bathing tool, the loofas are typically known as cylinders about 4 to 8 inches in length, with two flat, round ends having about 3 to 4 hollow openings traversing from one end to the other.

When preparing the matrix from the loofas, a first layer of loofas can be placed on the interior base (i.e., floor) of the tank, wherein the first layer comprises a plurality of rows of loofa sponges, said rows comprising a plurality of loofa sponges lined up in parallel to one another. Preferably the first layer covers the entire area of the tank base.

Next, a second layer can be stacked on top of the first layer, said second layer comprising a plurality of loofa sponges lined up in parallel to one another. Additional layers that are identical to the second layer are then stacked on top of the second layer until a plurality of layers is produced. The direction of the loofas is preferably the same for each row in a single layer (i.e., all in parallel), although the direction of alternating layers need not be in parallel. In other words, the loofas of one layer might be perpendicular to the loofas of the layer above and/or below it.

In an exemplary embodiment, the matrix can comprise from 15 to 50 layers. In another exemplary embodiment, one layer of loofa rows can comprise from 5 to 30 rows. In yet another exemplary embodiment, one row of loofas can comprise from 15 to 50 loofas. A greater or lesser number of layers, rows and/or loofas may be used, depending on the size of the tank.

Other materials, or pieces of materials, having similar layers, folds, crevices, holes, fibers, or other surfaces therein can also be used to form the matrix, such as synthetic loofas, natural or synthetic sponges, aquarium bio-balls, sea shells, mussel shells, conch shells, snail shells, scallop shells, and the like. These materials can be stacked in layers, or simply poured or placed into the system without any particular method of organization, depending upon, for example, the shape, size and uniformity of the material.

Preferably, the matrix reaches a height measuring about 50% to 90% of the tank height. If an impeller being used for mixing the height of the matrix must be low enough to stay clear of the rotating impeller.

Next, the method comprises submerging the fermentation matrix in liquid culture medium, wherein a liquid culture medium is added to the system using, for example, a peristaltic pump, and optionally, circulated through the system using the circulating pumps. In some embodiments, the amount of liquid culture medium added is an amount that covers the matrix entirely. Preferably, however, the culture medium does not fill the reactor vessel entirely, as additional liquid must be added to the system during the inoculation step.

In certain embodiments, the culture medium can include nutrient sources, for example, carbon sources, proteins (e.g., yeast extract or corn peptone), fats or lipids, nitrogen sources, trace elements, and/or growth factors (e.g., vitamins, pH regulators). Each of these nutrient sources can be provided in an individual package that can be added to the reactor at appropriate times during the fermentation process. Each of the packages can include several sub-packages that can be added at specific points (e.g., when microbe, pH, and/or nutrient levels go above or below a specific concentration) or times (e.g., after 10 hours, 20 hours, 30 hours, 40 hours, etc.) during the fermentation process. It will be apparent to one of skill in the art that nutrient concentration, moisture content, pH, and the like may be modulated to optimize growth for a particular microbe.

The lipid source can include oils or fats of plant or animal origin which contain free fatty acids or their salts or their esters, including triglycerides. Examples of fatty acids include, but are not limited to, free and esterified fatty acids containing from 16 to 18 carbon atoms, hydrophobic carbon sources, palm oil, animal fats, coconut oil, oleic acid, soybean oil, sunflower oil, canola oil, stearic and palmitic acid.

The carbon source is typically a carbohydrate, such as glucose, xylose, sucrose, lactose, fructose, trehalose, galactose, mannose, mannitol, sorbose, ribose, and maltose; organic acids such as acetic acid, fumaric acid, citric acid, propionic acid, malic acid, malonic acid, and pyruvic acid; alcohols such as ethanol, propanol, butanol, pentanol, hexanol, erythritol, isobutanol, xylitol, and glycerol; fats and oils such as canola oil, soybean oil, rice bran oil, olive oil, corn oil, sesame oil, and linseed oil; etc. Other carbon sources can include arbutin, raffinose, gluconate, citrate, molasses, hydrolyzed starch, potato extract, corn syrup, and hydrolyzed cellulosic material. The above carbon sources may be used independently or in a combination of two or more.

In one embodiment, growth factors and trace nutrients for microorganisms are included in the medium of the system. This is particularly preferred when growing microbes that are incapable of producing all of the vitamins they require. Inorganic nutrients, including trace elements such as iron, zinc, potassium, calcium copper, manganese, molybdenum and cobalt; phosphorous, such as from phosphates; and other growth stimulating components can be included in the culture medium of the subject systems. Furthermore, sources of vitamins, essential amino acids, and microelements can be included, for example, in the form of flours or meals, such as corn flour, or in the form of extracts, such as yeast extract, potato extract, beef extract, soybean extract, banana peel extract, and the like, or in purified forms. Amino acids such as, for example, those useful for biosynthesis of proteins, can also be included.

In one embodiment, inorganic or mineral salts may also be included. Inorganic salts can be, for example, potassium dihydrogen phosphate, dipotassium hydrogen phosphate, disodium hydrogen phosphate, magnesium sulfate, magnesium chloride, iron sulfate, iron chloride, manganese sulfate, manganese chloride, zinc sulfate, lead chloride, copper sulfate, calcium chloride, calcium carbonate, sodium carbonate. These inorganic salts may be used independently or in a combination of two or more.

The culture medium of the subject system can further comprise a nitrogen source. The nitrogen source can be, for example, in an inorganic form such as potassium nitrate, ammonium nitrate, ammonium sulfate, ammonium phosphate, ammonia, urea, and ammonium chloride, or an organic form such as proteins, amino acids, yeast extracts, yeast autolysates, corn peptone, casein hydrolysate, and soybean protein. These nitrogen sources may be used independently or in a combination of two or more.

In one embodiment, the culture medium can comprise agar and/or alginate, which can provide a semi-solid adherent for enhanced microbial deposition and adhesion onto the matrix. In certain embodiments, the matrix and the culture medium can be sterilized before and/or after addition to the system. This can be achieved using temperature decontamination and/or hydrogen peroxide decontamination (potentially followed by neutralizing the hydrogen peroxide using an acid such as HCl, H₂SO₄, etc.). In other embodiments, the culture medium may be pasteurized or optionally no heat at all added, where the use of low water activity and low pH may be exploited to control unwanted bacterial growth.

In a specific embodiment, the water used in the culture medium is UV sterilized using an in-line UV water sterilizer and filtered using, for example, a 0.1-micron water filter. In another embodiment, all nutritional and other medium components can be autoclaved prior to fermentation. The air can be sterilized by methods know in the art. For example, the ambient air can pass through at least one filter before being supplemented into the vessel.

To further prevent contamination, the culture medium of the system may comprise additional acids, antibiotics, and/or antimicrobials, which are added before, and/or during the cultivation process.

In one embodiment, the method further comprises inoculating the system with a microorganism. Preferably, inoculation according to the subject methods comprises mixing a microbial inoculant (e.g., cells and/or spores) in filtered water and pumping the inoculant and water into the system. The inoculant, water, and culture medium can then be circulated throughout the system to ensure exposure to the various surfaces of matrix and adhesion thereto. Circulation can be performed using, for example, the circulation pumps and external loops, or simply by using a mixing device.

In one embodiment, the method further comprises cultivating the microorganism for a number of days until a desired cell concentration is achieved. In one embodiment, the microorganisms grow for 1 to 21 days, preferably from 1 to 14 days, even more preferably from 2 to 10 days.

The fermenting temperature utilized in the subject systems and methods can be, for example, from about 20 to 40° C., although the process may operate outside of this range. In one embodiment, the method for cultivation of microorganisms is carried out at about 5° to about 100° C., preferably, 15° to 60° C., more preferably, 22 to 50° C. In a further embodiment, the cultivation may be carried out continuously at a constant temperature. In another embodiment, the cultivation may be subject to changing temperatures.

The pH of the medium should be suitable for the microorganism of interest. Buffering salts, and pH regulators, such as carbonates and phosphates, may be used to stabilize pH near an optimum value. When metal ions are present in high concentrations, use of a chelating agent in the liquid medium may be necessary.

In certain embodiments, the microorganisms can be fermented in a pH range from about 2.0 to about 10.0 and, more specifically, at a pH range of from about 3.0 to about 7.0 (by manually or automatically adjusting pH using bases, acids, and buffers; e.g., HCl, KOH, NaOH, H₂SO₄, and/or H₃PO₄). The invention can also be practiced outside of this pH range.

The fermentation can start at a first pH (e.g., a pH of 4.0 to 4.5) and later change to a second pH (e.g., a pH of 3.2-3.5) for the remainder of the process to help avoid contamination as well as to produce other desirable results (the first pH can be either higher or lower than the second pH). In some embodiments, pH is adjusted from a first pH to a second pH after a desired accumulation of biomass is achieved, for example, from 0 hours to 200 hours after the start of fermentation, more specifically from 12 to 120 hours after, more specifically from 24 to 72 hours after.

In one embodiment, the moisture level of the culture medium should be suitable for the microorganism of interest. In a further embodiment, the moisture level may range from 20% to 90%, preferably, from 30 to 80%, more preferably, from 40 to 60%.

The cultivation processes of the subject invention can be anaerobic, aerobic, or a combination thereof. Preferably, the process is aerobic, keeping the dissolved oxygen concentration above 10 or 15% of saturation during fermentation, but within 20% in some embodiments, or within 30% in some embodiments.

Advantageously, the system provides easy oxygenation of the growing culture with, for example, slow motion of air to remove low-oxygen containing air and introduction of oxygenated air. The oxygenated air may be ambient air supplemented periodically, such as daily.

Additionally, antifoaming agents can also be added to the system prevent the formation and/or accumulation of foam when gas is produced during cultivation.

The microbes can be grown in planktonic form or as biofilm. In the case of biofilm, the vessel may have within it a substrate upon which the microbes can be grown in a biofilm state. The system may also have, for example, the capacity to apply stimuli (such as shear stress) that encourages and/or improves the biofilm growth characteristics.

In one embodiment, the method further comprises harvesting the microorganisms from the system. In certain embodiments, harvesting comprises pumping a mixture comprising water and a biosurfactant into the system and optionally, circulating the mixture throughout the system. Preferably, the water is filtered water and the biosurfactant is a sophorolipid (SLP) at, e.g., 0.01 to 5 g/L, preferably, 0.1 g/L to 4 g/L.

In certain embodiments, the circulation of the mixture over and through the matrix provides enough agitation to detach the microorganisms from the matrix into the liquid. In certain embodiments, the SLP helps to break the surface tension between the microorganisms and the matrix, thus providing further detachment of microorganisms from the matrix. In certain embodiments, if further detachment is needed, air can be pumped into the system to provide turbulence to the circulating liquid.

After the microorganisms have detached, the liquid in the system, comprising detached microorganisms, growth by-products, water and residual nutrients, can be drained from the system. If needed, the harvesting process can be repeated until all or most of the culture has been detached from the matrix and washed from the system.

In one embodiment, the subject invention also provides methods of producing a microbial growth by-product, wherein the method comprises cultivating a microorganism according to the subject methods and under conditions favorable for growth and metabolite production, and optionally, purifying the growth by-product. In certain embodiments, the growth by-product is a biosurfactant, an enzyme, biopolymer, acid, solvent, amino acid, nucleic acid, peptide, protein, lipid and/or carbohydrate.

The subject invention further provides materials and methods for the production of biomass (e.g., viable cellular material), extracellular metabolites (e.g., both small and large molecules), and/or intracellular components (e.g., enzymes and other proteins).

In one embodiment, the subject invention provides a composition comprising at least one type of microorganism and/or at least one microbial metabolite produced by the microorganism. Preferably, the composition is produced according to the subject methods.

The microorganisms in the composition may be in an active or inactive form. The composition may or may not comprise the growth matrix in which the microbes were grown. The composition may also be in a dried form or a liquid form.

The composition can be subjected to filtration, centrifugation, lysing, drying or processing by any known means depending upon the desired use. Alternatively, the composition can be utilized as is, in liquid form, without further processing.

In one embodiment, the microbe-based composition does not need to be further processed after fermentation, however, if desired, the physical properties of the final product (e.g., viscosity, density, etc.) can also be adjusted using various chemicals and materials that are known in the art.

In one embodiment, the subject invention further provides customizations to the materials and methods according to the local needs. For example, the method for cultivation of microorganisms may be used to grow those microorganisms located in the local soil or at a specific oil well or site of pollution. In specific embodiments, local soils may be used as the solid substrates in the cultivation method for providing a native growth environment. Advantageously, these microorganisms can be beneficial and more adaptable to local needs.

Advantageously, the method and equipment of the subject invention reduce the capital and labor costs of producing microorganisms and their metabolites on a large scale. Furthermore, the subject invention provides a cultivation method that not only substantially increases the yield of microbial products per unit of nutrient medium but simplifies production in an environmentally-friendly manner using renewable substrates.

Advantageously, the method does not require complicated equipment or high energy consumption, and thus reduces the capital and labor costs of producing microorganisms and their metabolites on a large scale.

### Microorganisms

The microorganisms according to the subject invention can be strains of bacteria, yeast and/or fungi. These microorganisms may be natural, or genetically modified microorganisms. For example, the microorganisms may be transformed with specific genes to exhibit specific characteristics. The microorganisms may also be mutants of a desired strain. As used herein, “mutant” means a strain, genetic variant or subtype of a reference microorganism, wherein the mutant has one or more genetic variations (e.g., a point mutation, missense mutation, nonsense mutation, deletion, duplication, frameshift mutation or repeat expansion) as compared to the reference microorganism. Procedures for making mutants are well known in the microbiological art. For example, UV mutagenesis and nitrosoguanidine are used extensively toward this end.

In one embodiment, the beneficial microorganisms are yeasts and/or fungi. Yeast and fungus species suitable for use according to the current invention, include Acaulospora, Acremonium chrysogenum, Aspergillus, Aureobasidium (e.g., A. pullulans), Blakeslea, Candida (e.g., C. albicans, C. apicola, C. batistae, C. bombicola, C. floricola, C. kuoi, C. riodocensis, C. nodaensis, C. stellate), Cryptococcus, Debaryomyces (e.g., D. hansenii), Entomophthora, Hanseniaspora (e.g., H. uvarum), Hansenula, Issatchenkia, Kluyveromyces (e.g., K. phaffii), Lentinula spp. (e.g., L. edodes), Meyerozyma (e.g., M. guilliermondii), Monascus purpureus, Mortierella, Mucor (e.g., M. piriformis), Penicillium, Phythium, Phycomyces, Pichia (e.g., P. anomala, P. guilliermondii, P. occidentalis, P. kudriavzevii), Pleurotus (e.g., P. ostreatus P. ostreatus, P. sajorcaju, P. cystidiosus, P. cornucopias, P. pulmonarius, P. tuberregium, P. citrinopileatus and P. flabellatus), Pseudozyma (e.g., P. aphidis), Rhizopus, Rhodotorula (e.g., R. bogoriensis); Saccharomyces (e.g., S. cerevisiae, S. boulardii, S. torula), Starmerella hombicola), Torulopsis, Thraustochytrium, Trichoderma (e.g., T. reesei, T. harzianum, T. viride), Ustilago (e.g., U. maydis), Wickerhamiella (e.g., W. domericqiae), Wickerhamomyces (e.g., W. anomalus), Williopsis (e.g., W. mrakii), Zygosaccharomyces (e.g., Z. bailii), and others.

In certain embodiments, the microorganisms are bacteria, including Gram-positive and Gram-negative bacteria. The bacteria may be, for example Agrobacterium (e.g., A. radiobacter), Azotobacter (A. vinelandii, A. chroococcum), Azospirillum (e.g., A. brasiliensis), Bacillus (e.g., B. amyloliquefaciens, B. circulans, B. firmus, B. laterosporus, B. licheniformis, B. megaterium, Bacillus mucilaginosus, B. subtilis), Frateuria (e.g., F. aurantia), Microbacterium (e.g., M. laevaniformans), myxobacteria (e.g., Myxococcus xanthus, Stignatella aurantiaca, Sorangium cellulosum, Minicystis rosea), Pantoea (e.g., P. agglomerans), Pseudomonas (e.g., P. aeruginosa, P. chlororaphis subsp. aureofaciens (Kluyver), P. putida), Rhizobium spp., Rhodospirillum (e.g., R. rubrum), Sphingomonas (e.g., S. paucimobilis), and/or Thiobacillus thiooxidans (Acidothiobacillus thiooxidans).

In certain embodiments, the microorganisms are Trichoderma spp., Starmerella bombicola, Wickerhamomyces anomalus, Meyerozyma guilliermondii, Pichia spp., Saccharomyces cerevisiae, Lentinula edodes, Pleurotus ostreatus and/or Pseudozyma aphidis.

In some embodiments, the systems can be used for the production of bacteria-based compositions, including, for example, compositions comprising Bacillus spp., Pseudomonas spp., Rhodococcus spp., and/or Acinetobacter spp.

Other microbial strains including strains capable of accumulating significant amounts of, for example, glycolipid-biosurfactants (e.g., rhamnolipids, mannosylerythritol lipids and/or trehalose lipids), lipopeptide biosurfactants (e.g., surfactin, iturin, fengycin and/or lichenysin), mannoprotein, beta-glucan and other metabolites that have bio-emulsifying and surface/interfacial tension-reducing properties, can be used in accordance with the subject invention.

### Preparation of Microbe-Based Products

The microbe-based products of the subject invention include products comprising the microbes and/or microbial growth by-products and optionally, the growth medium and/or additional ingredients such as, for example, water, carriers, adjuvants, nutrients, viscosity modifiers, and other active agents.

One microbe-based product of the subject invention is simply the harvested liquid containing the microorganism and/or the microbial growth by-products produced by the microorganism and/or any residual nutrients. The product of fermentation may be used directly without extraction or purification. If desired, extraction and purification can be easily achieved using standard extraction methods or techniques known to those skilled in the art.

The microorganisms in the microbe-based products may be in an active or inactive form and/or in the form of vegetative cells, spores, mycelia, conidia and/or any form of microbial propagule. The microbe-based products may be used without further stabilization, preservation, and storage. Advantageously, direct usage of these microbe-based products preserves a high viability of the microorganisms, reduces the possibility of contamination from foreign agents and undesirable microorganisms, and maintains the activity of the by-products of microbial growth.

The microbes and/or medium resulting from the microbial growth can be removed from the growth vessel and transferred via, for example, piping for immediate use.

In other embodiments, the composition (microbes, medium, or microbes and medium) can be placed in containers of appropriate size, taking into consideration, for example, the intended use, the contemplated method of application, the size of the fermentation tank, and any mode of transportation from microbe growth facility to the location of use. Thus, the containers into which the microbe-based composition is placed may be, for example, from 1 gallon to 1,000 gallons or more. In other embodiments the containers are 2 gallons, 5 gallons, 25 gallons, or larger.

Upon harvesting the microbe-based composition from the growth vessels, further components can be added as the harvested product is placed into containers and/or piped (or otherwise transported for use). The additives can be, for example, buffers, carriers, other microbe-based compositions produced at the same or different facility, viscosity modifiers, preservatives, nutrients for microbe growth, nutrients for plant growth, tracking agents, pesticides, herbicides, animal feed, food products and other ingredients specific for an intended use.

Advantageously, in accordance with the subject invention, the microbe-based product may comprise broth in which the microbes were grown. The product may be, for example, at least, by weight, 1%, 5%, 10%, 25%, 50%, 75%, or 100% broth. The amount of biomass in the product, by weight, may be, for example, anywhere from 0% to 100% inclusive of all percentages there between.

Optionally, the product can be stored prior to use. The storage time is preferably short. Thus, the storage time may be less than 60 days, 45 days, 30 days, 20 days, 15 days, 10 days, 7 days, 5 days, 3 days, 2 days, 1 day, or 12 hours. In a preferred embodiment, if live cells are present in the product, the product is stored at a cool temperature such as, for example, less than 20° C., 15° C., 10° C., or 5° C. On the other hand, a biosurfactant composition can typically be stored at ambient temperatures.

The compositions produced according to the present invention have advantages over purified microbial metabolites alone, including: high concentrations of emulsifiers such as mannoprotein as a part of yeast cell wall's outer surface and beta-glucan in yeast cell walls; the presence of biosurfactants in the culture; and the presence of metabolites (e.g., lactic acid, ethanol, etc.) in the culture. These compositions can, among many other uses, act as biosurfactants and can have surface/interfacial tension-reducing properties.

The subject invention further provides microbe-based products, as well as uses for these products to achieve beneficial results in many settings including, for example, improved bioremediation, mining, and oil and gas production; waste disposal and treatment; enhanced health of livestock and other animals; and enhanced health and productivity of plants by applying one or more of the microbe-based products. The microbe-based products may be, for example, microbial inoculants, biopesticides, nutrient sources, remediation agents, health products, and/or biosurfactants.

In one embodiment, the cultivation broth and/or biomass may be dried (e.g., spray-dried), to produce the products of interest. The biomass may be separated by known methods, such as centrifugation, filtration, separation, decanting, a combination of separation and decanting, ultrafiltration or microfiltration.

In one embodiment, the biomass cultivation products may be used as an animal feed or as food supplement for humans. Thus, the biomass cultivation products may be further treated to facilitate rumen bypass. The biomass product may be separated from the cultivation medium, spray-dried, and optionally treated to modulate rumen bypass, and added to feed as a nutritional source.

In one embodiment, the cultivation products have a high nutritional content. As a result, a higher percentage of the cultivation products may be used in a complete animal feed. In one embodiment, the feed composition comprises the modified cultivation products ranging from 15% of the feed to 100% of the feed. The cultivation products may be rich in at least one or more of fats, fatty acids, lipids such as phospholipid, vitamins, essential amino acids, peptides, proteins, carbohydrates, sterols, enzymes, and trace minerals such as, iron, copper, zinc, manganese, cobalt, iodine, selenium, molybdenum, nickel, fluorine, vanadium, tin and silicon. The peptides may contain at least one essential amino acid.

In other embodiments, the essential amino acids are encapsulated inside a subject modified microorganism used in a cultivation reaction. The essential amino acids are contained in heterologous polypeptides expressed by the microorganism. Where desired, the heterologous peptides are expressed and stored in the inclusion bodies in a suitable microorganism (e.g., fungi).

The microbes and microbial growth by-products of the subject invention can also be used for the transformation of a substrate, such as an ore, wherein the transformed substrate is the product.

In one embodiment, the composition is suitable for agriculture. For example, the composition can be used to treat soil, plants, and seeds. The composition may also be used as a pesticide. In specific embodiments, the systems of the subject invention provide science-based solutions that improve agricultural productivity by, for example, promoting crop vitality; enhancing crop yields; enhancing plant immune responses; enhancing insect, pest and disease resistance; controlling insects, nematodes, diseases and weeds; improving plant nutrition; improving the nutritional content of agricultural and forestry and pasture soils; and promoting improved and more efficient water use.

In one embodiment, the subject invention provides a method of improving plant health and/or increasing crop yield by applying the composition disclosed herein to soil, seed, or plant parts. In another embodiment, the subject invention provides a method of increasing crop or plant yield comprising multiple applications of the composition described herein.

Advantageously, the method can effectively control nematodes, and the corresponding diseases caused by pests while a yield increase is achieved and side effects and additional costs are avoided.

1. A system for producing a microorganism and/or a growth by-product thereof, the system comprising:
a fermentation vessel;
an internal mixing device;
an external circulation system;
a sparging system; and
a programmable logic controller (PLC) to monitor and adjust fermentation parameters,
wherein said external circulation system also functions as a temperature control system.

2. A method for producing a microorganism and/or a growth by-product thereof, wherein said method comprises:
preparing a fermentation matrix comprised of a solid material in the vessel of the system of claim 1;
submerging the fermentation matrix in a liquid culture medium;
inoculating the system with a microorganism;
cultivating the microorganism in the system; and
harvesting the microorganism and any growth by-products produced during cultivation.

3. The method of claim 2, wherein the system is sterilized prior to preparing the fermentation matrix, after preparing the fermentation matrix and/or after the fermentation matrix is submerged in the liquid culture medium.

4-5. (canceled)

6. The method of claim 2, wherein the fermentation matrix is prepared by placing or pouring a solid material, or pieces of a solid material, into the vessel until the amount of solid material or pieces thereof reaches a height within the tank that measures from 50% to 90% of the tank height.

7. The method of claim 6, wherein the solid material comprises synthetic loofas, natural or synthetic sponges, aquarium bio-balls, sea shells, mussel shells, conch shells, snail shells, or scallop shells.

8. The method of claim 2, wherein submerging the matrix in liquid culture medium comprises pumping a culture medium into the system.

9. The method of claim 2, wherein the culture medium comprises one or more protein, carbon, lipid and/or nitrogen sources.

10. The method of claim 9, wherein the culture medium further comprises agar and/or alginate.

11. The method of claim 2, wherein inoculating the system comprises mixing cells of a microorganism with filtered water, and circulating the cells, water and culture medium through the system, wherein the cells adhere to the matrix.

12. The method of claim 11, wherein the microorganism is a yeast, fungus or bacterium.

13. The method of claim 12, wherein the microorganism is Wickerhamomyces anomalus, Starmerella bombicola, Saccharomyces cerevisiae, Saccharomyces boulardii, Pseudozyma aphidis, Meyerozyma guilliermondii, Pichia kudriavzevii, Trichoderma harzianum, Pleurotus ostreatus, Lentinula edodes, Bacillus subtilis, Bacillus amyloliquefaciens, Bacillus megaterium, Bacillus licheniformis, Rhodococcus erythropolis, Acinetobacter vinelandii, or Pseudomonas chlororaphis.

14. The method of claim 2, wherein the microorganism is cultivated for 1 to 14 days.

15. The method of claim 2, wherein harvesting the microorganism comprises pumping a mixture of filtered water and a biosurfactant into the system and circulating the water and biosurfactant through the system to detach the microorganism from the matrix.

16. The method of claim 15, wherein the biosurfactant is a sophorolipid (SLP).

17. The method of claim 16, further comprising pumping air into the system to create turbulence in the liquid.

18. The method of claim 15, wherein harvesting further comprises draining all liquid from the system, wherein the liquid in the system comprises the water and biosurfactant mixture, the microorganisms and any growth by-products thereof, and residual culture medium.

19. The method of claim 2, wherein the harvesting is repeated until all of the microorganism has been collected from the system.

20. (canceled)
